(19) United States
(12) Patent Application Publication
(10) Pub. No.: US 2007/0259041 A1
(43) Pub. Date: Nov. 8, 2007

(54) Solid dosage formulations

(75) Inventors: Christopher Richard Diorio, Campbell Hall, NY (US); Eric Ehrnsperger, New City, NY (US); Syed M. Shah, East Hanover, NJ (US)

Correspondence Address: HOWSON AND HOWSON/WYETH;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 11/799,870

(22) Filed: May 3, 2007

Related U.S. Application Data
(60) Provisional application No. 60/798,272, filed on May 5, 2006.

(51) Int. Cl. A61K 31/137; A61K 9/24

(52) U.S. Cl. 424/472; 514/650

(57) ABSTRACT

Solid dosage formulations are provided for a compound having the formula:

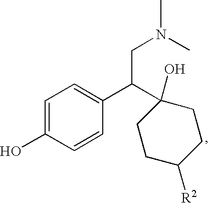

wherein R² is Cl, F, Br, CH₃, CF₃, SCH₃, NHCH₃, NO₂, CN,
OH, OC₁—C₆ alkyl, or substituted OC₁—C₆ alkyl,
or a prodrug or a pharmaceutically acceptable salt thereof.

Formulations for tablets and multiparticulates containing a compound according to the above formula, a rate controlling component, and a binder are described, including formulations containing a seal coating, release rate controlling coating, and/or enteric coating. Pharmaceutical uses and kits thereof are also described.

### CROSS-REFERENCE TO RELATED APPLICATIONS

This application claims the benefit under 35 USC 119(e) of prior U.S. Provisional Patent Application No. 60/798,272, filed May 5, 2006.

### BACKGROUND OF THE INVENTION

The market for neuroscience and women's health drugs has been moving towards the use of dual serotonin and norepinephrine reuptake inhibitors (SNRI) for first line treatment of various indications, as evidence by the recent development of SNRIs, for example, Venlafaxine and Duloxetine. This contrasts with the traditional use of selective serotonin reuptake inhibitors (SSRI). Although the side-effect profile of SSRIs and SNRIs are less severe as compared to older, tricyclic antidepressant compounds, there are still some undesirable side effects.

What are needed are alternative compositions for treating conditions associated with serotonin and norepinephrine imbalances.

### SUMMARY OF THE INVENTION

In one aspect, the invention provides modified release formulations having a tablet core containing a compound of formula I (shown below), or a prodrug or a pharmaceutically acceptable salt thereof; at least one rate controlling component; at least one binder; and at least one lubricant.

In another aspect, the invention provides are multiparticulate modified release formulations, where each multiparticulate has a spheroid core containing a compound of formula I, or a prodrug or a pharmaceutically acceptable salt thereof; at least one rate controlling component; and at least one binder.

In still another aspect, the invention provides multiparticulate formulations, where the multiparticulates have a seal coating and/or a release rate controlling coating and/or an enteric coating applied to a tablet or multiparticulate core.

In a further embodiment, the invention provides capsules containing multiparticulates described herein. Foil packets comprising the multiparticulates are also provided.

In yet another embodiment, the invention provides the use of the composition described herein in the preparation of medicaments for an array of indications.

Still other aspects and advantages of the invention will be apparent from the following detailed description of the invention.

### DETAILED DESCRIPTION OF THE INVENTION

The present invention provides pharmaceutical compositions comprising a modified release dosage form of an active compound of formula I, shown below.

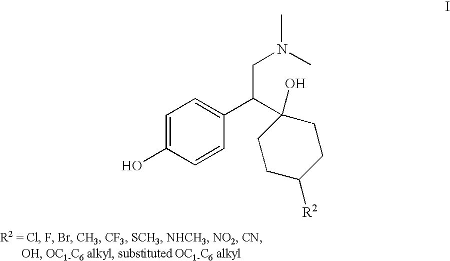

, or a prodrug or a pharmaceutically acceptable salt thereof.

### Advantageously, these formulations alleviate upper gastrointestinal adverse effects associated with dual serotonin and norepinephrine reuptake inhibitors (SNRI).

While not wishing to be bound by theory, these formulations are believed to be effective by reducing the interaction of the active compound with neuro-receptors in the stomach and small intestine as well as systemically. Accordingly, slower (extended) release or enteric coating products with minimal release in the stomach serve to minimize the concentration of the active compound in the upper gastrointestinal tract.

An extended release (release rate controlling) or enterically coated dosage form has the advantages of reducing adverse upper gastrointestinal effects, for example, nausea and vomiting by limiting the amount of drug release there and by-passing the receptors in the upper gastrointestinal tract that cause these effects. Additionally, a once daily dosed dosage form is expected to result in improved patient compliance over multiple dosing.

### I. Active Compounds

The compounds of formula I (above), and methods for the preparation thereof, are described in US Published Patent Application No. US-2007-0015828-A1, published Jan. 18, 2007 (U.S. patent application Ser. No. 11/485,663, Jul. 13, 2006, claiming priority of U.S. Provisional Patent Application No. 60/699,665, Jul. 15, 2005), which are hereby incorporated by reference. As described therein, the compounds of formula I (above) may contain one or more asymmetric carbon atoms and some of the compounds may contain one or more asymmetric (chiral) centers and may thus give rise to optical isomers and diastereomers. While shown without respect to stereochemistry in formula I, in one embodiment, carbon 1 is present as a chiral center. However, this molecule can exist in a form of R and S isomers as well as in racemic mixture. There are also two diasteromers. The two groups on the cyclohexane ring could be in the cis or trans configuration, but in one embodiment are in the cis configuration. For example, in one embodiment, the compound is in a configuration greater than 50% cis diasteromer. In another embodiment, the compound is in a configuration greater than 95% cis diastereomer. Thus, the compound of formula I includes such optical isomers and diastereomers; as well as the racemic and resolved, enantiomerically pure stereoisomers; as well as the other mixtures of the R and S stereoisomers, and pharmaceutically acceptable salts, hydrate, and prodrugs thereof.

The term “alkyl” is used herein to refer to both straight- and branched-chain saturated aliphatic hydrocarbon groups, generally of 1, 2, 3, 4, 5, 6, 7 or 8 carbon atoms in length, unless otherwise specified. The term “lower alkyl” is used to refer to alkyl chains of 1, 2, 3, or 4 carbons in length. The terms “substituted alkyl” refers to alkyl as just described having from one to three substituents selected from the group including halogen, CN, OH, NO₂, amino, aryl, heterocyclic, substituted aryl, substituted heterocyclic, alkoxy, aryloxy, substituted alkyloxy, alkylcarbonyl, alkylcarboxy, alkylamino, arylthio. These substituents may be attached to any carbon of alkyl group provided that the attachment constitutes a stable chemical moiety.

The term “halogen” refers to Cl, Br, F, or I.

The term “aryl” is used herein to refer to a carbocyclic aromatic system, which may be a single ring, or multiple aromatic rings fused or linked together as such that at least one part of the fused or linked rings forms the conjugated aromatic system. The aryl groups include, but are not limited to, phenyl, naphthyl, biphenyl, anthryl, tetrahydronaphthyl, and phenanthryl.

The term “substituted aryl” refers to aryl as just defined having one, two, three or four substituents from the group including halogen, CN, OH, NO₂, amino, alkyl, cycloalkyl, alkenyl, alkynyl, alkoxy, aryloxy, substituted alkyloxy, alkylcarbonyl, alkylcarboxy, alkylamino, and arylthio.

The term “heterocyclic” is used herein to describe a stable 4-, 5-, 6- or 7-membered monocyclic or a stable multicyclic heterocyclic ring which is saturated, partially unsaturated, or unsaturated, and which consists of carbon atoms and from one to four heteroatoms selected from the group including N, O, and S atoms. The N and S atoms may be oxidized. The heterocyclic ring also includes any multicyclic ring in which any of above defined heterocyclic rings is fused to an aryl ring. The heterocyclic ring may be attached at any heteroatom or carbon atom provided the resultant structure is chemically stable. Such heterocyclic groups include, for example, tetrahydrofuran, piperidinyl, piperazinyl, 2-oxopiperidinyl, azepinyl, pyrrolidinyl, imidazolyl, pyridyl, pyrazinyl, pyrimidinyl, pyridazinyl, oxazolyl, isoxazolyl, morpholinyl, indolyl, quinolinyl, thienyl, furyl, benzofuranyl, benzothienyl, thiamorpholinyl, thiamorpholinyl sulfoxide, and isoquinolinyl.

The term “substituted heterocyclic” is used herein to describe the heterocyclic just defined having one to four substituents selected from the group which includes halogen, CN, OH, NO₂, amino, alkyl, substituted alkyl, cycloalkyl, alkenyl, substituted alkenyl, alkynyl, alkoxy, aryloxy, substituted alkyloxy, alkylcarbonyl, alkylcarboxy, alkylamino, or arylthio.

The term “alkoxy” is used herein to refer to the OR group, where R is alkyl or substituted alkyl. The substituted alkoxy may be the OR group where R is C₁, C₂, C₃, C₄, C₅ or C₆ alkyl substituted by from one to three substituents selected from the group including halogen, CN, OH, NO₂, amino, aryl, heterocyclic, substituted aryl, substituted heterocyclic, alkoxy, aryloxy, substituted alkyloxy, alkylcarbonyl, alkylcarboxy, alkylamino, arylthio. The term “aryloxy” is used herein to refer to the OR group, where R is aryl or substituted aryl. The term “alkylcarbonyl” is used herein to refer to the RCO group, where R is alkyl or substituted alkyl. The term “alkylcarboxy” is used herein to refer to the COOR group, where R is alkyl or substituted alkyl. The term “aminoalkyl” refers to both secondary and tertiary amines wherein the alkyl or substituted alkyl groups, containing one to eight carbon atoms, which may be either same or different and the point of attachment is on the nitrogen atom.

The compounds of formula I can be used in the form of salts derived from pharmaceutically or physiologically acceptable acids or bases. These salts include, but are not limited to, the following salts with organic and inorganic acids, for example, acetic, lactic, citric, tartaric, succinic, fumaric, maleic, malonic, mandelic, mallic, hydrochloric, hydrobromic, phosphoric, nitric, sulfuric, methanesulfonic, toluenesulfonic and similarly known acceptable acids, and mixtures thereof. Other salts include salts with alkali metals or alkaline earth metals, for example, sodium (e.g., sodium hydroxide), potassium (e.g., potassium hydroxide), calcium or magnesium.

These salts, as well as other compounds of formula I, may be in the form of esters, carbamates and other conventional “pro-drug” forms, which, when administered in such form, convert to the active moiety in vivo. In a currently preferred embodiment, the prodrugs are esters. See, e.g., B. Testa and J. Caldwell, “Prodrugs Revisited: The “Ad Hoc” Approach as a Complement to Ligand Design”, Medicinal Research Reviews, 16(3):233-241, ed., John Wiley & Sons (1996).

As used herein, the term “about” generally means within 5%, 1%, or 0.5% of a given value or range. Alternatively, the term “about” means within an acceptable standard error of the mean, when considered by one of ordinary skill in the art. Further, the total of all components contained within a core (either tablet or multiparticulate) for any particular formulation does not exceed 100% of the core.

### II. Tablets

Provided herein are modified release formulations having a tablet core containing a compound of formula I, or a prodrug or a pharmaceutically acceptable salt thereof; at least one rate controlling component; at least one binder; and at least one lubricant.

In one embodiment, the tablet core contains from about 10% to about 30% (weight by weight (w/w) of the tablet core) of a compound of formula I. In a further embodiment, the tablet core contains from about 10% to about 15% w/w, about 10% to about 20% w/w, about 10% to about 25% w/w, about 15% to about 20% w/w, about 15% to about 25% w/w, about 15% to about 30% w/w, about 20% to about 25% w/w, about 20% to about 30% w/w, or about 25% to about 30% w/w of a compound of formula I. In still another embodiment, the tablet core contains about 15% to about 16%, about 16% to about 17%, or about 21% to about 22% w/w of a compound of formula I. In yet another embodiment, the tablet core contains from about 15% to about 16% w/w of a compound of formula I. In another embodiment, the tablet core contains from about 16% to about 17% w/w of a compound of formula I. In another embodiment, the tablet core contains from about 21% to about 22% w/w of a compound of formula I.

In one embodiment, the rate controlling component is a rate controlling polymer selected from among hydrophilic polymers and inert plasticized polymers. Suitable rate controlling hydrophilic polymers include, without limitation, polyvinyl alcohol (PVA), hydroxypropyl methylcellulose (HPMC, hypomellose or hypromellose), and mixtures thereof. Suitable insoluble or inert “plastic” polymers include, without limitation, one or more polymethacrylates (i.e., Eudragit® polymer and equivalent polymers under other trademarks). Other suitable rate controlling polymer materials include, e.g., hydroxyalkyl celluloses, poly(ethylene) oxides, alkyl celluloses, carboxymethyl celluloses, hydrophilic cellulose derivatives, and polyethylene glycol. In one embodiment, the rate controlling component is hydroxypropyl methylcellulose.

Suitably, the tablet core contains from about 30% to about 50% (weight by weight (w/w) of the tablet core) of a rate controlling component. In a further embodiment, the tablet core contains from about 30% to about 35% w/w, about 30% to about 40% w/w, about 30% to about 45% w/w, about 35% to about 40% w/w, about 35% to about 45% w/w, about 35% to about 50% w/w, about 40% to about 45% w/w, about 40% to about 50% w/w, or about 45% to about 50% w/w of a rate controlling component. In still another embodiment, the tablet core contains about 38% to about 42% w/w, about 42% to about 43% w/w, or about 43% to about 44% w/w. In still a further embodiment, the tablet core contains 40% w/w of a rate controlling component.

The binder may be selected from among known binders, including, e.g., cellulose, and povidone, among others. In one embodiment, the binder is selected from among microcrystalline cellulose, crospovidone, and mixtures thereof. In a further embodiment, the binder is microcrystalline cellulose, and optionally Avicel® microcrystalline cellulose or Avicel® PH101 microcrystalline cellulose.

Suitably, the tablet core contains from about 25% to about 50% (weight by weight (w/w) of the tablet core) of a binder. In a further embodiment, the tablet core contains from about 25% to about 30% w/w, about 25% to about 35% w/w, about 25% to about 40% w/w, about 25% to about 45% w/w, about 30% to about 35% w/w, about 30% to about 40% w/w, about 30% to about 45% w/w, about 30% to about 50% w/w, about 35% to about 40% w/w, about 35% to about 45% w/w, about 35% to about 50% w/w, about 40% to about 45% w/w, about 40% to about 50% w/w, or about 45% to about 50% w/w of a binder. In still another embodiment, the tablet core contains about 26% to about 27% w/w, about 32-33% w/w, or about 43-44% w/w of a binder.

The lubricant may be selected from any of the conventional lubricants known to those of skill in the art for tablet formulations. In one embodiment, the lubricant is magnesium stearate. In one embodiment, the tablet core contains about 0.5% to about 3% w/w, or about 1% to about 3% w/w, of a lubricant. In a further embodiment, the tablet core contains about 1% w/w of a lubricant.

In one embodiment, other components including diluents (e.g., magnesium stearate), fillers, glidants (e.g., talc), anti-adherents, pH adjusters and/or adjuvants may be included in the tablet core. In a further embodiment, the tablet core contains from about 5% to about 10% w/w of a glidant. In one embodiment, the glidant is talc. Suitable pH adjusters include, e.g., sodium carbonate, sodium bicarbonate, potassium carbonate, lithium carbonate, among others. Still other suitable components will be readily apparent to one of skill in the art. See, e.g., R. Rowe, et al., Handbook of Pharmaceutical Excipients, Fourth Edition, Pharmaceutical Press, London, United Kingdom (2003), which is hereby incorporated by reference.

In one embodiment, a modified release formulation has a tablet core containing:
about 15% to about 16% w/w of the tablet core of a compound of formula I; or a prodrug or a pharmaceutically acceptable salt thereof;
about 40% w/w of the tablet core of a rate controlling component;
about 43% to about 44% w/w of the tablet core of a binder; and
about 1% w/w of the tablet core of a lubricant.

In a further embodiment, a modified release formulation has a tablet core containing:
about 15% w/w of a compound of formula I, or a prodrug or a pharmaceutically acceptable salt thereof;
about 40% w/w of hydroxypropyl methylcellulose;
about 44% w/w of microcrystalline cellulose (for example, Avicel® microcrystalline cellulose); and
about 1% w/w of magnesium stearate.

In another embodiment, a modified release formulation has a tablet core containing:
about 16% to about 17% w/w of the tablet core of a compound of formula I, or a prodrug or a pharmaceutically acceptable salt thereof;
about 43% to about 44% w/w of the tablet core of a rate controlling component;
about 32% to about 33% w/w of the tablet core of a binder; and
about 8% to about 9% w/w of the tablet core of a lubricant.

In a further embodiment, the modified release formulation also contains about 7% to about 9% w/w (solid, weight gain) of the tablet core of a release rate controlling coating (described below) over the tablet core.

In still a further embodiment, the modified release formulation has a tablet core containing:
about 16% w/w of the tablet core of the compound of formula I, or a prodrug or a pharmaceutically acceptable salt thereof;
about 44% w/w of the tablet core of hydroxypropyl methylcellulose;
about 32% w/w of the tablet core of microcrystalline cellulose;
about 6% w/w of the tablet core of talc;
about 2% w/w of the tablet core of magnesium stearate; and
a release rate controlling coating over the tablet core comprising:
about 7% w/w (solid, weight gain) of the tablet core of ethylcellulose with plasticizer (for instance, using Surelease® ethylcellulose dispersion (25% w/w aqueous dispersion)); and
about 0.6% w/w of the tablet core of hydroxypropyl methylcellulose.

In still another embodiment, the modified release formulation has a tablet core containing:
about 21% to about 22% w/w of the tablet core of a compound of formula I, or a prodrug or a pharmaceutically acceptable salt thereof;
about 42% to about 43% w/w of the tablet core of a rate controlling component;
about 26% to about 27% w/w of the tablet core of a binder; and
about 10% to about 11% w/w of the tablet core of a lubricant.

In a further embodiment, the modified release formulation also contains about 17% to about 18% w/w (solid, weight gain) of the tablet core of an enteric coating (described below) over the tablet core.

In a further embodiment, the modified release formulation has a tablet core containing:
about 21% w/w of the tablet core of a compound of formula I, or a prodrug or a pharmaceutically acceptable salt thereof;
about 42% w/w of the tablet core of hydroxypropyl methylcellulose;
about 26% w/w of the tablet core of microcrystalline cellulose;
about 8% w/w of the tablet core of talc;
about 3% w/w of the tablet core of magnesium stearate; and

an enteric coating over the tablet core comprising:
about 14% w/w (solid, weight gain) of the tablet core of methacrylic acid copolymer type C;
about 0.5% w/w of the tablet core of triethyl citrate;
about 0.7% w/w of the tablet core of sodium hydroxide; and
about 2% w/w of the tablet core of talc.

Tablets may be prepared by conventional methods known in the art. In one embodiment, a compound of formula I is mixed with the other components of the formulation to form a granulation. In one embodiment, the granulation is formed using a roller compactor. In another embodiment, the granulation is formed using a high shear granulator (e.g., a Collette Gral mixer). However, other methods known to those of skill in the art, including, e.g., a low shear granulator, a blender, planetary mixer, etc., or a fluid bed processor (Glatt GPCG), dry granulation, or slugging, can be utilized to prepare suitable granulations. The granulation is then compressed using conventional methods to form a tablet.

Tablets may be provided with additional layers, optionally, containing active components, or other layers as may be desired for coatings (as described below), separation between layers, or the like.

### III. Multiparticulates

Provided herein are multiparticulate modified release formulations, wherein each multiparticulate has a spheroid core containing a compound of formula I, or a prodrug or a pharmaceutically acceptable salt thereof; at least one rate controlling component; and at least one binder.

In one embodiment, the multiparticulate core contains from about 15% to about 35% (weight by weight (w/w) of the multiparticulate core) of a compound of formula I. In a further embodiment, the multiparticulate core contains from about 15% to about 20% w/w, about 15% to about 25% w/w, about 15% to about 30% w/w, or about 20% to about 25% w/w, about 20% to about 30% w/w, about 20% to about 35% w/w, about 25% to about 30% w/w, about 25% to about 35% w/w, or about 30% to about 35% w/w of a compound of formula I. In still another embodiment, the multiparticulate core contains about 23% to about 24% w/w of a compound of formula I.

In one embodiment, the rate controlling component is a rate controlling polymer selected from among hydrophilic polymers and inert plasticized polymers. Suitable rate controlling hydrophilic polymers include, without limitation, polyvinyl alcohol (PVA), hydroxypropyl methylcellulose (HPMC, hypomellose or hypromellose), and mixtures thereof. Suitable insoluble or inert “plastic” polymers include, without limitation, one or more polymethacrylates (i.e., Eudragit® polymer and equivalent polymers under other trademarks). Other suitable rate controlling polymer materials include, e.g., hydroxyalkyl celluloses, poly(ethylene) oxides, alkyl celluloses, carboxymethyl celluloses, hydrophilic cellulose derivatives, and polyethylene glycol. In one embodiment, the rate controlling component is hydroxypropyl methylcellulose.

Suitably, the multiparticulate core contains from about 20% to about 40% (weight by weight (w/w) of the multiparticulate core) of a rate controlling component. In a further embodiment, the multiparticulate core contains from about 20% to about 25% w/w, about 20% to about 30% w/w, about 20% to about 35% w/w, about 25% to about 30% w/w, about 25% to about 35% w/w, about 25% to about 40% w/w, about 30% to about 35% w/w, about 30% to about 40% w/w, or about 35% to about 40% w/w of a rate controlling component. In still another embodiment, the multiparticulate core contains about 30% to about 31% w/w of a rate controlling component.

The binder may be selected from among known binders, including, e.g., cellulose, and povidone, among others. In one embodiment, the binder is selected from among microcrystalline cellulose, crospovidone, and mixtures thereof. In a further embodiment, the binder is microcrystalline cellulose, and optionally Avicel® microcrystalline cellulose or Avicel® PH101 microcrystalline cellulose.

Suitably, the multiparticulate core contains from about 35% to about 55% (weight by weight (w/w) of the multiparticulate core) of a binder. In a further embodiment, the multiparticulate core contains from about 35% to about 40% w/w, about 35% to about 45% w/w, about 35% to about 50% w/w, about 40% to about 45% w/w, about 40% to about 50% w/w, about 40% to about 55% w/w, about 45% to about 50% w/w, about 45% to about 55% w/w, or about 50% to about 55% w/w of a binder. In still another embodiment, the multiparticulate core contains about 46% to about 47% w/w of a binder.

In one embodiment, other components including lubricants (e.g., magnesium stearate), diluents, fillers, glidants (e.g., talc), anti-adherents, pH adjusters and/or adjuvants may be included in the tablet core. Suitable pH adjusters include, e.g., sodium carbonate, sodium bicarbonate, potassium carbonate, lithium carbonate, among others. Still other suitable components will be readily apparent to one of skill in the art. See, e.g., R. Rowe, et al., Handbook of Pharmaceutical Excipients, Fourth Edition, Pharmaceutical Press, London, United Kingdom (2003), which is hereby incorporated by reference.

In one embodiment is a multiparticulate modified release formulation, wherein each multiparticulate has a spheroid core containing:
about 23% to about 24% w/w of the multiparticulate core of a compound of formula I, or a prodrug or a pharmaceutically acceptable salt thereof;
about 30% to about 31% w/w of the multiparticulate core of a rate controlling component; and
about 46% to about 47% w/w of the multiparticulate core of a binder.

In a further embodiment, a seal coat (described below) is applied over the multiparticulate core of about 1% to about 2% w/w of the multiparticulate core.

In still a further embodiment, an enteric coat (described below) is applied over the multiparticulate core and seal coat of about 8% to about 9% w/w (solid, weight gain) of the multiparticulate core.

In a further embodiment is a multiparticulate modified release formulation, wherein each multiparticulate has a spheroid core containing:
about 23% w/w of the multiparticulate core of a compound of formula I, or a prodrug or a pharmaceutically acceptable salt thereof;
about 30% w/w of the multiparticulate core of hydroxypropyl methylcellulose;
about 46% w/w of the multiparticulate core of microcrystalline cellulose;

a seal coating over the multiparticulate core containing about 1% w/w of the multiparticulate core of a seal coat comprising hydroxypropyl methylcellulose with polyethylene glycol as plasticizer (for example, an Opadry® Clear seal coating); and

an enteric coating over the multiparticulate core, the enteric coating containing:
about 7% w/w (solid, weight gain), of the multiparticulate core of ethylcellulose with plasticizer (for instance, using Surelease® ethylcellulose dispersion (25% w/w aqueous dispersion)); and
about 1% w/w of the multiparticulate core of hydroxypropyl methylcellulose.

Multiparticulate formulations may be prepared by methods known in the art. In one embodiment, the dry components, including at least the compound of formula I and the binder are dry blender in a suitable mixer, e.g., a planetary mixer, for example, a Hobart mixer. Optionally the rate controlling component, and further optionally a pH adjuster, may be included in this step. Subsequently, the remaining components and water are mixed in to afford a granulated product. The granulation is then extruded and spheronized through a suitable device (e.g., a Nica® extruder/spheronizer) and the resulting spheroids are dried, sifted and optionally blended to generate the multiparticulate formulations.

In another embodiment, the multiparticulate formulation components are granulated with water in a suitable mixer, e.g., a planetary mixer, for example, a Hobart mixer. Then, using the Nica® system, the resulting wet mass is extruded through a 1 mm or 1.0 mm screen. The extrudates are then transferred to a spheronizer and spun until spherical pellets are obtained (approximately 2-3 minutes). In one embodiment, the extrudates are spun at approximately 700 rpm. The wet pellets are then dried in an Aeromatic Strea™ fluid bed dryer to a moisture level of 2% to 5%. The dried pellets are then passed through a mesh screen to remove larger, i.e., oversize, pellets to provide multiparticulate formulations. In one embodiment, an 18 mesh screen is used.

Multiparticulates, either uncoated or coated as described below, may be placed into a capsule shell, compressed into tablets or caplets, or packaged in a foil packet or other suitable package, and are suitable for mixing into a food product (e.g., applesauce or the like).

### IV. Coatings

In one aspect, a seal coat can be applied to the uncoated tablet or multiparticulate. This may serve as an initial seat coat, as a final seal coat (i.e., over all other coatings applied), or both. The seal coating may be selected from among suitable polymers, for example, hydroxypropyl methylcellulose (HPMC, hypomellose or hypromellose), ethylcellulose, polyvinyl alcohol, and combinations thereof, optionally containing plasticizers and other desirable components. In one embodiment, the seal coat is HPMC. In another embodiment, the seal coat comprises hydroxypropyl methylcellulose with polyethylene glycol as plasticizer. Such a seal coat may be made from Opadry® Clear coating. The seal coat is applied to provide the desired weight gain to the tablet or multiparticulate. In one embodiment, the coating is applied to a 0.5%-3% (w/w), 0.5%, 1%, 2%, or 3% w/w weight gain (solid) with respect to the uncoated form. One of skill in the art will be able to apply a seal coating as described herein by conventional methods known in the art. However, the means for applying any of the coatings described herein is not a limitation of the invention.

In another embodiment, an initial seal coat can be applied to multiparticulates on a fluid bed coater, e.g., by spraying. In one embodiment, an Aeromatic Strea™ fluid bed apparatus is fitted with a Wurster column and bottom spray nozzle system. An appropriate amount as determined by the capacity of the system, and in one embodiment approximately 200 grams, of the dried pellet cores (multiparticulates) are charged into the unit. The coating, e.g., Opadry® Clear seal coat, is then applied under conventional conditions, and dried. In one embodiment, the coating is applied with an inlet temperature of approximately 50° C. to 60° C., a coating solution spray rate of 5 to 10 grams per minute, and atomization pressure of 1 to 2 bar. In one embodiment, the multiparticulate temperature is 35° C. to 45° C., or about 38° C. to about 43° C. Optionally, talc or a comparable material is applied to the finally coated formulation.

In another aspect, an extended release or release rate controlling coat is applied, optionally in addition to any of the other coatings described herein. The extended release coating layer may be applied over an initial seal coat, over an enteric coat, or directly over a core. This coating is applied by the same means as described above. In one embodiment, the release coat is obtainable from an ethylcellulose-based product and HPMC. An example of one suitable ethylcellulose-based product is an aqueous ethylcellulose dispersion (25% solids). One such product is commercially available as Surelease® ethylcellulose dispersion (Colorcon, Inc.). In one embodiment, a solution of an aqueous ethylcellulose (25% solids) dispersion is applied to the core. In one embodiment, HPMC, e.g., in an amount of about 5% to 15% w/w, or about 10% w/w, is mixed with the ethylcellulose dispersion, to form the coat solution. Thus, the ethylcellulose may be about 85% to about 95% w/w, or about 90% w/w, of the coat solution. Upon drying under suitable conditions, e.g., approximately an additional 5 to 10 minutes, the total release coat is in the range of about 1% to about 10%, 2% to about 9%, 3% to about 8%, or about 8% to about 9% w/w of the core prior to application of this coating, i.e., including any prior coats.

In yet another aspect, an enteric coat is applied, optionally in addition to any of the other coatings described herein. The enteric coating layer may be applied over an initial seal coat, over an extended release coat, or directly over a core. This coating is applied by the same means as described above. In one embodiment, the enteric coat applied to the tablet or multiparticulate may include, but is not limited to, polymethacrylates, HPMC, ethylcellulose, or a combination thereof.

In a further embodiment, the enteric coat contains a product which is a copolymer containing units of a monomer selected from methacrylic acid and methacrylates, for example, methacrylic acid copolymer, Type C, USP (which is a copolymer of methacrylic acid and ethyl acrylate). Such a copolymer is commercially available in the form of an aqueous dispersion with 30% dry substance as Eudragit® L30-D55 (Röhm GmbH & Co. KG). The dry substance itself comprising the copolymer is available as Eudragit® L 100-55 (as a powder). The dispersion and powder contain 0.7% sodium laurylsulfate and 2.3% Polysorbate 80, calculated on the dry substance, as emulsifiers. Kollicoat MAE 30 DP (from BASF) is another example of an aqueous dispersion of methacrylic acid copolymer type C. In still a further embodiment of the invention, the enteric coat applied is composed of methacrylic acid copolymer, Type C, USP/NF (for instance, Eudragit® L 100-55 copolymer), triethyl citrate, talc (or other comparable), and water (subject to drying). In a further embodiment, a pH adjuster, for example, sodium hydroxide is part of the coating. While not so limited, the enteric coat may be prepared from about 70% to 90% w/w of aqueous copolymer dispersion (containing 30% dry matter and 70% water), 1% to 5% w/w triethyl citrate, 1% to 10% w/w pH adjuster, and 5% to 15% w/w talc (or other comparable material) as a percentage of the weight of starting materials. In a further embodiment, the enteric coat may be prepared from 80% w/w of aqueous copolymer dispersion containing 30% dry matter and 70% water (for example, Eudragit® L30-D55 copolymer dispersion), 3% w/w triethyl citrate, 4% w/w sodium hydroxide, and 12% w/w talc as a percentage of the weight of starting materials.

Upon drying under suitable conditions, e.g., approximately an additional 5 to 10 minutes, the total enteric coat is in the range of about 10% to about 30% w/w, 15% to about 25% w/w, or about 17% to about 23% w/w of the uncoated or initially coated tablet or multiparticulate, i.e., including any prior coats. In a further embodiment, the coating is about 17% to about 18%, or about 17.71% of the core tablet or multiparticulate.

In still another embodiment, one or more of the coating layers contain a compound of formula I. Coated multiparticulates may be screened to remove agglomerates and oversize particles following application of any coating layer.

### V. Kits/Packs

Also encompassed by the invention are pharmaceutical packs and kits comprising a container, for example, a foil package or other suitable container, for multiparticulates, tablets, capsules, or caplets as described herein. In a further embodiment, the kit or pack contains instructions for use of the multiparticulates, tablets, capsules, or caplets.

### VI. Use of the Formulations, Kits, and Packs

Formulations of the invention are useful in treating, and in preparing medicaments useful in the treatment of, indications such as Irritable Bowel Syndrome (IBS), where the higher norepinephrine (NE) activity of SNRIs limits the application because of constipation side effects. These formulations are also expected to be effective in applications where reduction of histamine side effects are desired.

The formulations of the present invention can be used to treat or prevent central nervous system disorders including, but not limited to, depression (including but not limited to, major depressive disorder, bipolar disorder and dysthymia), fibromyalgia, anxiety, panic disorder, agoraphobia, post traumatic stress disorder, premenstrual dysphoric disorder (also known as premenstrual syndrome), attention deficit disorder (with or without hyperactivity), obsessive compulsive disorder (including trichotillomania), social anxiety disorder, generalized anxiety disorder, autism, schizophrenia, obesity, anorexia nervosa, bulimia nervosa, Gilles de la Tourette Syndrome, vasomotor flushing, cocaine and alcohol addiction, sexual dysfunction, (including premature ejaculation), borderline personality disorder, chronic fatigue syndrome, incontinence (including fecal incontinence, overflow incontinence, passive incontinence, reflex incontinence, stress urinary incontinence, urge incontinence, urinary exertional incontinence and urinary incontinence), pain (including but not limited to migraine, chronic back pain, phantom limb pain, central pain, neuropathic pain, for example, diabetic neuropathy, and postherpetic neuropathy), Shy Drager syndrome, Raynaud's syndrome, Parkinson's Disease, epilepsy, and others. Formulations of the present invention can also be used for preventing relapse or recurrence of depression; to treat cognitive impairment; for the inducement of cognitive enhancement in patient suffering from senile dementia, Alzheimer's disease, memory loss, amnesia and amnesia syndrome; and in regimens for cessation of smoking or other tobacco uses. Additionally, formulations of the present invention can be used for treating hypothalamic amenorrhea in depressed and non-depressed human females.

Other uses for the formulations of the invention will be known by one of ordinary skill in the art, and are intended to be encompassed by the present invention.

The following examples are provided to illustrate the invention and do not limit the scope thereof. One skilled in the art will appreciate that although specific amounts, components, and conditions are outlined in the following examples, modifications can be made which are meant to be encompassed by the spirit and scope of the invention.

### EXAMPLES

### Example 1—Tablet

A tablet having a compound of formula I is prepared according to the following table.

TABLE I

| Ingredient | % w/w | mg/tablet |
|---|---|---|
| Compound of formula I | 15.38% | 50.00 |
| HPMC 2208 K100 | 40.00% | 130.00 |
| Avicel PH101 | 43.62% | 141.75 |
| Magnesium Stearate | 1.00% | 3.25 |
| | 100.00% | 325.00 |

A. Synthesis of Compound of Formula I

A compound of formula I is prepared by Scheme I or Scheme II (see below), as described in US Published Patent Application No. US-2007-0015828-A1, published Jan. 18, 2007, which is hereby incorporated by reference, together with synthetic methods known in the synthetic organic arts or variations of these methods by one skilled in the art. [See, generally, Comprehensive Organic Synthesis, “Selectivity, Strategy & Efficiency in Modern Organic Chemistry”, ed., I. Fleming, Pergamon Press, New York (1991); Comprehensive Organic Chemistry, “The Synthesis and Reactions of Organic Compounds”, ed. J. F. Stoddard, Pergamon Press, New York (1979)].

### Scheme 1

The following scheme illustrates the synthesis of a compound of formula I. A similar method is used for synthesis of the other compounds of formula I using different intermediates with the appropriate groups. These intermediates are commercially available.

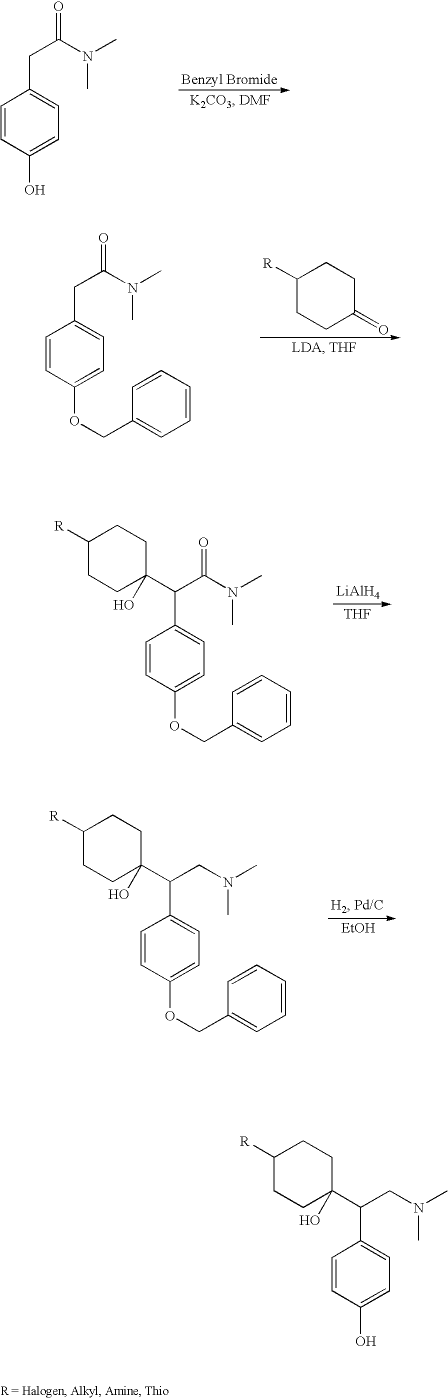

### Scheme 2

The following scheme illustrates the synthesis of a compound of formula I, where R²═O(R).

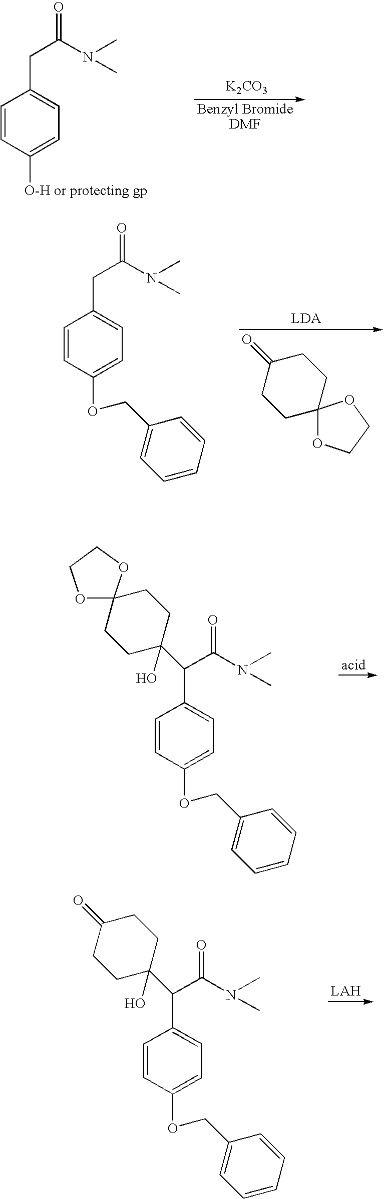

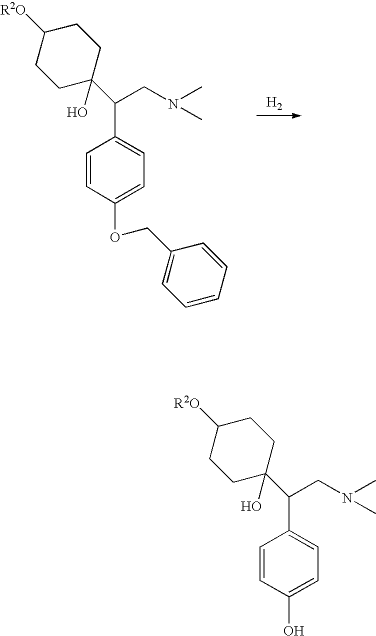

4-(Dimethylcarbamoylmethyl)phenol in dimethylformamide (DMF) is treated with K₂CO₃ followed by benzyl bromide.

The mixture is stirred at room temperature followed by heating at 60° C. for 1 hour. The mixture is concentrated to remove DMF, diluted with EtOAc and washed with water. Dry MgSO₄ is added, the mixture filtered and concentrated to low volume. Hexane is added to precipitate the ketal intermediate product. Solids are collected via filtration and dried.

A solution of the 1,4-cyclohexanedione-mono-ethylene ketal in 100 mL THF/50 mL MeOH is treated with acid (e.g., HCl), then stirred at room temperature. Where R is other than O(H, substituted or unsubstituted alkyl), the corresponding R group is added to the ketal either before the LDA reaction or after the LDA reaction using conventional methods. The reaction is quenched with saturated K₂CO₃, extracted with EtOAc and concentrated to an oil. Product is crystallized from hot EtOAc/hexanes to provide the ketone intermediate.

A solution of the ketone in THF was added to a suspension of lithium aluminum hydride (LAH) pellets in THF at −78° C. The mixture is warmed to room temperature and stirred for at least 3 hours. The reaction is quenched with MeOH followed by 10% NaOH and stirred for at least 3 hours. The solid are removed by filtration, followed by a wash (e.g., with THF), and concentrated to give a solid. The resulting solid is recrystallized from EtOAc/hexanes to provide the corresponding benzyl ether.

A mixture of the benzyl ether and Pd/C in 100 mL of ethanol are hydrogenated under pressure overnight. The solid is purified by filtration followed by an ethanol wash. Solid is concentrated and crystallized from EtOAc/hexane to give the final product.

Salts for use in the formulations are formed by contacting stoichiometric amounts of the acid with the free base. The crystalline salt is prepared by directly crystallizing from a solvent.

B. Formulation

The compound of formula I, a portion of the microcrystalline cellulose, the hydroxypropyl methylcellulose (HPMC), and a portion of the magnesium stearate are blended together and then dry granulated via roller compaction. The resulting compacts are then sized by milling and/or screening. The remaining microcrystalline cellulose is blended in and the granulation is lubricated with the remaining magnesium stearate and compressed into tablets.

### Example 2—Extended Release Coated Tablet

An extended release coated tablet having a compound of formula I is prepared according to the following table.

TABLE II

| Ingredient | mg/capsule |
|---|---|
| Tablet Core: | |
| Compound of formula I | 50.00 |
| hydroxypropyl methylcellulose | 135.00 |
| Microcrystalline cellulose | 100.00 |
| talc | 18.00 |
| magnesium stearate | 7.00 |
| ER Coat: | |
| Surelease ® ethylcellulose | 23.0* |
| Dispersion | |
| hydroxypropyl methylcellulose | 2.0 |
| Water | N/A** |

*Reflects dry weight of solids
**Does not appear in final formula

The tablet core is prepared as described above for the tablet of Example 1. The ethylcellulose is applied using a fluid bed apparatus fitted with a Wurster column and bottom spray nozzle system. The components for making the extended release (ER) coating are combined and applied to the tablet with an inlet temperature of approximately 60° C., a coating solution spray rate of 5-10 grams/minute, and atomization pressure of 1-2 bar. The desired tablet temperature is 38° C. to 43° C. After the appropriate weight gain, the coated tablet is dried for approximately 5 to 10 minutes.

### Example 3—Enteric Coated Tablet

An enteric coated tablet having a compound of formula I is prepared according to the following table.

TABLE III

| Ingredient | mg/capsule |
|---|---|
| Tablet Core: | |
| Compound of formula I | 50.00 |
| hydroxypropyl methylcellulose | 100.00 |
| Microcrystalline cellulose | 62.00 |
| talc | 18.00 |
| magnesium stearate | 7.00 |
| Enteric Coat: | |
| Eudragit L30-D55 | 34.00* |
| Triethyl Citrate | 1.2 |
| Sodium Hydroxide | 1.75 |
| Talc | 5.00 |

*Reflects dry weight of solids

The tablet core is prepared as described above for the tablet of Example 1. The components for making the enteric coat are combined and applied as indicated for the extended release coat in Example 2.

### Example 4—Multiparticulate

A multiparticulate having a compound of formula I is prepared according to the following table.

TABLE IV

| Ingredient | mg/capsule |
|---|---|
| Pellet Core: | |
| Compound of formula I | 50.0 |
| Microcrystalline cellulose | 100.0 |
| Hydroxypropyl methylcellulose | 65.0 |
| Seal Coat: | |
| Opadry ® Clear seal coating | 2.50 |
| Release Rate Controlling Coat: | |
| Surelease ® ethylcellulose | 16.0* |
| dispersion | |
| hydroxypropylmethylcellulose | 2.0 |
| Water | NA** |

*Reflects dry weight of solids
**Does not appear in final formula

The compound of formula I is combined with microcrystalline cellulose and/or HPMC and granulated with water in a planetary mixer. Then using the Nica® System, the resulting wet mass is extruded through a 11.0 mm screen. The extrudates are then transferred to the spheronizer and spun for approximately 2-3 minutes at approximately 700 rpm until spherical pellets are obtained.

The wet pellets are then dried in a fluid bed dryer to a moisture level of 2-5%. The dried pellets are passed through a 18 mesh screen to remove larger oversize pellets.

The fluid bed apparatus is fitted with a Wurster column and bottom spray nozzle system. The Opadry® seal coat is applied with a inlet temperature of approximately 60° C., a coating solution spray rate of 5-10 grams/minute, atomization pressure of 1-2 bar. The desired product temperature is 38° C.-43° C. After the appropriate weight gain of the seal coat is achieved the ethylcellulose coat can be applied.

The ethylcellulose and hydroxypropylmethylcellulose are applied in a similar fashion as the seal coat to the appropriate weight gain. After the coat comprising ethylcellulose and hydroxypropylmethylcellulose is applied, the pellets are dried for an additional 5-10 minutes. They are removed and screened through an 18-mesh screen to remove agglomerates and oversize particles.

All documents listed in this specification are incorporated herein by reference. While the invention has been described with reference to a particularly preferred embodiment, it will be appreciated that modifications can be made without departing from the spirit of the invention. Such modifications are intended to fall within the scope of the appended claims.

1. A modified release formulation having a tablet core comprising:
a compound of the structure: 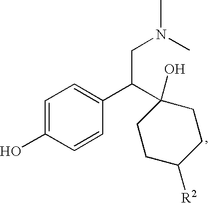
wherein R² is Cl, F, Br, CH₃, CF₃, SCH₃, NHCH₃, NO₂, CN,
OH, OC₁—C₆ alkyl, or substituted OC₁—C₆ alkyl,
or a prodrug or a pharmaceutically acceptable salt thereof;
at least one rate controlling component;
at least one binder; and
at least one lubricant.

2. The formulation according to claim 1, further comprising a coating over said tablet core.

3. The formulation according to claim 2, wherein said coating is a seal coating.

4. The formulation according to claim 3, wherein said seal coating is a release rate controlling coating.

5. The formulation according to claim 4, wherein said coating comprises ethylcellulose.

6. The formulation according to claim 5, wherein said coating is ethylcellulose with plasticizer.

7. The formulation according to claim 5, wherein said coating further comprises hydroxypropyl methylcellulose.

8. The formulation according to claim 2, wherein said coating is an enteric coating.

9. The formulation according to claim 8, wherein said coating comprises a copolymer containing units of a monomer selected from methacrylic acid and methacrylates.

10. The formulation according to claim 9, wherein said copolymer is methacrylic acid copolymer, Type C.

11. The formulation according to claim 8, wherein said enteric coating also contains triethyl citrate.

12. The formulation according to claim 1, wherein said rate controlling component is hydroxypropyl methylcellulose.

13. The formulation according to claim 1, wherein said binder is microcrystalline cellulose.

14. The formulation according to claim 13, wherein said microcrystalline cellulose is Avicel® microcrystalline cellulose.

15. The formulation according to claim 1, wherein said lubricant is magnesium stearate.

16. A multiparticulate modified release formulation, wherein each said multiparticulate comprises a spheroid core comprising:
a compound of the structure: 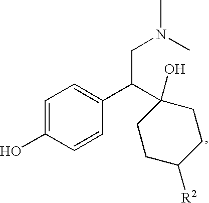
wherein R² is Cl, F, Br, CH₃, CF₃, SCH₃, NHCH₃, NO₂, CN,
OH, OC₁—C₆ alkyl, or substituted OC₁—C₆ alkyl,
or a prodrug or a pharmaceutically acceptable salt thereof;
at least one rate controlling component; and
at least one binder.

17. The formulation according to claim 16, further comprising a seal coating over said multiparticulate core.

18. The formulation according to claim 17, wherein said seal coating comprises hydroxypropyl methylcellulose.

19. The formulation according to claim 17, wherein said seal coating comprises hydroxypropyl methylcellulose with polyethylene glycol as plasticizer.

20. The formulation according to claim 17, further comprising a release rate controlling coating.

21. The formulation according to claim 20, wherein said release rate controlling coating comprises ethylcellulose.

22. The formulation according to claim 21, wherein said release rate controlling coating further comprises hydroxypropyl methylcellulose.

23. The formulation according to claim 21, wherein said release rate controlling coating comprises ethylcellulose with plasticizer.

24. The formulation according to claim 16, further comprising an enteric coating over said multiparticulate core.

25. The formulation according to claim 24, wherein said enteric coating comprises methacrylic acid copolymer, Type C.

26. The formulation according to claim 16, wherein said rate controlling component is hydroxypropyl methylcellulose.

27. The formulation according to claim 16, wherein said binder is microcrystalline cellulose.

28. The formulation according to claim 1, wherein R² of said compound is OH.

29. The formulation according to claim 1, wherein R² of said compound is O-methyl.

30. A modified release formulation having a tablet core comprising:
about 15% to about 16% w/w of the tablet core of a compound of the structure: 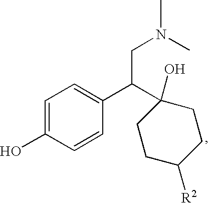
wherein R² is Cl, F, Br, CH₃, CF₃, SCH₃, NHCH₃, NO₂, CN,
OH, OC₁—C₆ alkyl, or substituted OC₁—C₆ alkyl,
or a prodrug or a pharmaceutically acceptable salt thereof;
about 40% w/w of the tablet core of a rate controlling component;
about 43% to about 44% w/w of the tablet core of a binder; and
about 1% w/w of the tablet core of a lubricant.

31. The formulation according to claim 30, comprising:
15.38% w/w of said compound, or a prodrug or a pharmaceutically acceptable salt thereof;
40% w/w of hydroxypropyl methylcellulose;
43.62% w/w of microcrystalline cellulose; and
1% w/w of magnesium stearate.

32. A modified release formulation having a tablet core comprising:
about 16% to about 17% w/w of the tablet core of a compound of the structure: 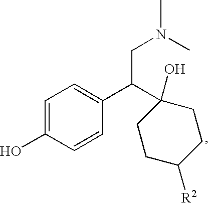
wherein R² is Cl, F, Br, CH₃, CF₃, SCH₃, NHCH₃, NO₂, CN,
OH, OC₁—C₆ alkyl, or substituted OC₁—C₆ alkyl,
or a prodrug or a pharmaceutically acceptable salt thereof;
about 43% to about 44% w/w of the tablet core of a rate controlling component;
about 32% to about 33% w/w of the tablet core of a binder; and
about 8% to about 9% w/w of the tablet core of a lubricant.

33. The formulation according to claim 32, further comprising a release rate controlling component over the tablet core comprising about 8% to about 9% w/w of the tablet core.

34. The formulation according to claim 32, comprising:
16.13% w/w of the tablet core of said compound, or a prodrug or a pharmaceutically acceptable salt thereof;
43.55% w/w of the tablet core of hydroxypropyl methylcellulose;
32.26% w/w of the tablet core of microcrystalline cellulose;
5.81% w/w of the tablet core of talc;
2.26% w/w of the tablet core of magnesium stearate; and
a release rate controlling coating over the tablet core comprising:
7.42% w/w of the tablet core of ethylcellulose with plasticizer; and
0.65% w/w of the tablet core of hydroxypropyl methylcellulose.

35. A modified release formulation having a tablet core comprising:
about 21% to about 22% w/w of the tablet core of a compound of the structure: 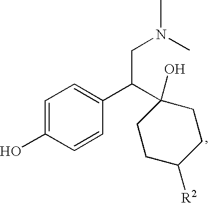
wherein R² is Cl, F, Br, CH₃, CF₃, SCH₃, NHCH₃, NO₂, CN,
OH, OC₁—C₆ alkyl, or substituted OC₁—C₆ alkyl,
or a prodrug or a pharmaceutically acceptable salt thereof;
about 42% to about 43% w/w of the tablet core of a rate controlling component;
about 26% to about 27% w/w of the tablet core of a binder; and
about 10% to about 11% w/w of the tablet core of a lubricant.

36. The formulation according to claim 35, further comprising an enteric coating over the tablet core comprising about 17% to about 18% w/w of the tablet core.

37. The formulation according to claim 35, comprising:
21.10% w/w of the tablet core of said compound, or a prodrug or a pharmaceutically acceptable salt thereof;
42.19% w/w of the tablet core of hydroxypropyl methylcellulose;
26.16% w/w of the tablet core of microcrystalline cellulose;
7.59% w/w of the tablet core of talc;
2.95% w/w of the tablet core of magnesium stearate; and
an enteric coating over the tablet core comprising:
14.35% w/w of the tablet core of methacrylic acid copolymer type C;
0.51% w/w of the tablet core of triethyl citrate;
0.74% w/w of the tablet core of sodium hydroxide; and
2.11% w/w of the tablet core of talc.

38. A multiparticulate modified release formulation, wherein each said multiparticulate comprises a spheroid core comprising:
about 23% to about 24% w/w of the tablet core of a compound of the structure: 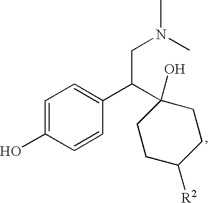
wherein R² is Cl, F, Br, CH₃, CF₃, SCH₃, NHCH₃, NO₂, CN,
OH, OC₁—C₆ alkyl, or substituted OC₁—C₆ alkyl,
or a prodrug or a pharmaceutically acceptable salt thereof;
about 30% to about 31% w/w of the multiparticulate core of a rate controlling component; and
about 46% to about 47% w/w of the multiparticulate core of a binder.

39. The formulation according to claim 38, further comprising a seal coating over the multiparticulate core comprising about 1% to about 2% w/w of the multiparticulate core.

40. The formulation according to claim 38, further comprising an enteric coating over the multiparticulate core comprising about 8% to about 9% w/w of the multiparticulate core.

41. The formulation according to claim 38, comprising:
23.26% w/w of the multiparticulate core of said compound, or a prodrug or a pharmaceutically acceptable salt thereof;
30.23% w/w of the multiparticulate core of hydroxypropyl methylcellulose;
46.51% w/w of the multiparticulate core of microcrystalline cellulose;
a seal coating over the multiparticulate core comprising:
1.16% w/w of the multiparticulate core of a seal coating comprising hydroxypropyl methylcellulose with polyethylene glycol as plasticizer; and
an enteric coating over the multiparticulate core comprising:
7.44% w/w of the multiparticulate core of ethylcellulose with plasticizer; and
0.93% w/w of the multiparticulate core of hydroxypropyl methylcellulose.

42. A capsule comprising the multiparticulates claim 16.

43. A foil packet comprising the multiparticulates of claim 16.

44. A method of treating irritable bowel syndrome in a mammal in need thereof, which comprises providing to said mammal an effective amount of a formulation according to claim 1.
